(19) United States
(12) Patent Application Publication
(10) Pub. No.: US 2007/0260314 A1
(43) Pub. Date: Nov. 8, 2007

(54) TRANSFORAMINAL LUMBAR INTERBODY FUSION CAGE

(76) Inventor: Ashok Biyani, Toledo, OH (US)

Correspondence Address: WOOD, HERRON & EVANS (ZIMMER SPINE), 2700 CAREW TOWER, 441 VINE STREET, CINCINNATI, OH, 45202, US

(21) Appl. No.: 11/742,873

(22) Filed: May 1, 2007

Related U.S. Application Data
(60) Provisional application No. 60/796,691, filed on May 2, 2006.

(51) Int. Cl. A61F 2/44

(52) U.S. Cl. 623/17.11

(57) ABSTRACT

A cage to separate and support adjacent vertebrae in the spine that have undergone orthopedic spinal fusion procedures. The cage has first and second spacer members for insertion between adjacent vertebrae with a hinge located between the spacers. An advancing mechanism is located between the first and second spacer members that pivotally moves the first and second spacer members relative to each other at an angle which facilitates the insertion of the cage around the spinal cord. After insertion, the advancing mechanism is operable to position the first and second spacer members in the desired position between the two adjacent vertebrae.

The present application claims the filing benefit of U.S. Provisional Application Ser. No. 60/796,691, filed May 2, 2006, the disclosure of which is hereby incorporated herein by reference in its entirety.

### FIELD OF THE INVENTION

The present invention relates generally to the field of orthopedic surgery and, more particularly, to the area of spinal implants.

### BACKGROUND OF THE INVENTION

Fusion cages have generally been used in orthopedic surgery for fixing bones in a pre-selected spacial orientation. However, in inserting such fusion cages using minimally invasive surgical techniques, it is oftentimes difficult to insert a fusion cage without making an incision that is larger than desired or significantly displacing the neural element. Typically, interbody fusion cages of the prior art require considerable space to be rotated into the proper position between adjacent vertebrae. To properly position such prior art cages it was generally necessary to make a larger incision or displace the nerve roots more than desirable, or both, to properly position the fusion cage. To overcome the limitations of prior fusion cages, the present invention utilizes an articulated fusion cage that can be displaced during the insertion process to move around the neural element in a manner that takes less room. This facilitates insertion of the cage during minimally invasive spinal surgery and reduces the need to displace the spinal cord more than is desirable. As the fusion cage of the present invention is maneuvered into position, the angular relationship between the two portions of the cage can be adjusted so that the cage is in the proper orientation when finally inserted.

### SUMMARY OF THE INVENTION

The present invention overcomes the foregoing and other shortcomings and drawbacks of the interbody fusion cages heretofore known. While the invention will be described in connection with certain embodiments, it will be understood that the invention is not limited to these embodiments. On the contrary, the invention includes all alternatives, modifications and equivalents as may be included within the spirit and scope of the present invention.

The present invention relates to a fusion cage that is used to separate and support adjacent vertebrae in the spine. The fusion cage may be designed for use in the lumbar region of the spine, although it is possible to use the fusion cage of the present invention in other areas of the spine as well. The fusion cage has a first spacer member or chamber and a second spacer member or chamber that are pivotally interconnected by an articulating mechanism such as a hinge. The first and second spacer members are designed for insertion between adjacent vertebrae to properly support and separate the vertebrae. An advancing mechanism is located between the first and second spacer members to pivotally move the first spacer member relative to the second spacer member around the hinge. The angular position of the first spacer member relative to the second spacer member facilitates the insertion of the fusion cage around the dural sac and reduces the space necessary for the insertion of the cage. The advancing mechanism is operable to adjust the angular position of the first and second spacer members so that the first and second spacer members are in the desired position relative to the adjacent vertebrae when the cage is fully inserted.

The above and other objects and advantages of the present invention shall be made apparent from the accompanying drawings and the description thereof.

### BRIEF DESCRIPTION OF THE DRAWINGS

The accompanying drawings, which are incorporated in and constitute a part of this specification, illustrate embodiments of the invention and, together with a general description of the invention given above, and the detailed description of the embodiments given below, serve to explain the principles of the invention.

FIG. 1 is a top view showing a fusion cage according to one embodiment of the present invention in an open or expanded position.

FIG. 2 is a top view showing the fusion cage of FIG. 1 in a closed or collapsed position.

FIG. 3 is a right side view of the fusion cage in a collapsed position.

### DETAILED DESCRIPTION OF THE PREFERRED EMBODIMENT(S)

The present invention is directed to an interbody fusion cage that is used in spinal fusion procedures, such as a transforaminal lumbar spinal fusion procedure, by way of example. More particularly, the present invention is directed to an articulated fusion cage that can be adjusted in configuration to facilitate the insertion of the cage between adjacent vertebrae in the spine, such as the lumbar region. The fusion cage of the present invention may be inserted by the use of minimally invasive surgical techniques wherein relatively small incisions are made in the patient and instruments are utilized to guide the cage to the desired location between adjacent vertebrae. The articulated nature of the cage allows the cage to be disposed at an angle that facilitates the insertion of the cage around the neural elements and reduces the displacement or impact on the nerve roots during the insertion process.

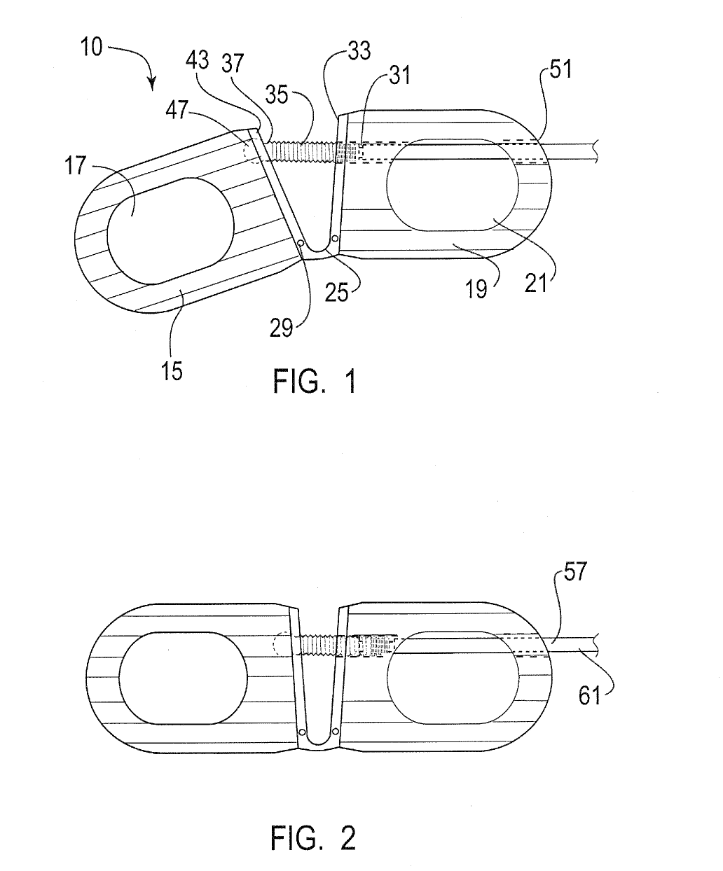

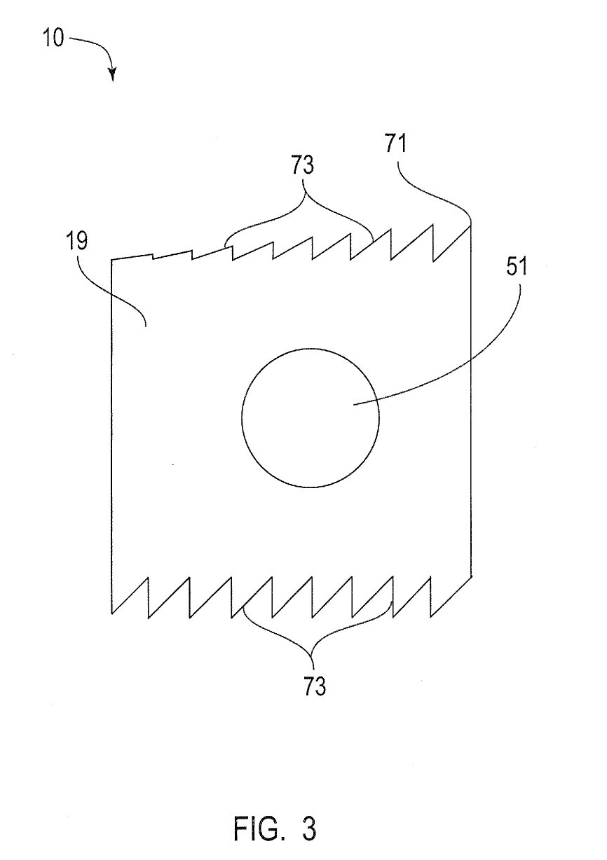

Referring now to the figures, and to FIGS. 1-2 in particular, the fusion cage 10 has a first spacer member or chamber 15 and a second spacer member or chamber 19 that are connected together by an articulating mechanism, such as hinge 25, to form the complete fusion cage. The cage may be made of reinforced carbon fiber, PEEK polymer material, titanium or other suitable biomaterial. The hinge 25 may be made of nitinol, titanium or other material. The hinge may incorporate holes 29 in the hinge material to assist in connecting to the cage material. The holes 29 provide an opening in which the cage material can protrude to form a secure bond between the cage and the hinge.

Alternatively, the hinge 25 could be created by using a mechanism similar to one seen in a door hinge, wherein one chamber of the fusion cage pivots in relation to the other. The two chambers 15, 19 of the fusion cage 10 interdigitate at the hinge 25 allowing them to pivot in relation to each other. It will be appreciated that other types of hinge or articulating mechanisms known to those of ordinary skill in the art are possible as well without departing from the spirit and scope of the present invention.

In one embodiment, the first space member 15 and second spacer member 19 may be generally elliptical in shape when looked at from above and openings 17 and 21 may be provided in first and second spacer members, respectively, as shown in FIGS. 1-2. In one embodiment, the fusion cage 10 may have a lordotic shape wherein the front of the cage 10 is taller than the back. The cage 10 may have serrations 73 provided on the top and bottom of the cage. The first and second spacer members 15, 19 of the cage 10 may be the same length or may vary in size with the second spacer member 19 being longer and making up to 70% of the total length of the cage 10. The first and second spacer members 15, 19 may be designed to fit between and properly space adjacent vertebrae in the lumbar region of the spine. The fusion cage 10 may be used when a disk is removed from between the vertebrae and it is necessary to use the cage 10 to provide the necessary spacing between the vertebrae and to stabilize the vertebrae after the disc has been removed. In most applications, bone or bone graft substitute will be positioned in the openings 17, 21 of the first and second spacer members 15, 19 so that the bone can fuse with the adjacent vertebrae to complete the repair on the spine.

In one embodiment, a threaded passageway 31 extends from the opening 21 in the second spacer member 19 to the end 33 of the second spacer member 19 that is adjacent to the first spacer member 15. The threaded passageway 31 may be metallic and made of material such as nitinol or titanium. The passageway 31 may be encased within the wall of the trailing chamber 19. An advancement mechanism, such as a threaded rod/screw 35, may be positioned in the threaded passageway 31 so that the threads on the rod engage the threads on the threaded passageway 31. The end 37 of the rod 35 that is spaced apart from the opening 21 in the second spacer member 19 is disposed to engage an edge 43 of the first spacer member 15. A pivoting foot or ball in socket 47 design may be employed on the end of the threaded rod 35 that engages the edge 43 of the first spacer member 15. The pivoting foot or ball and socket design facilitates angulation of the cage as the hinge is deployed. A port 51 may extend through a portion of the second spacer member 19 that is on the opposite side of the opening 21 from the threaded passageway 31. The port 51 may extend into the opening 21 and is disposed to be in alignment with the threaded passageway 31. The port 51 may be threaded to facilitate placement of a cage inserter or tool 57 having a shaft 61 as shown in FIG. 2, which can be inserted into the port 51 and advanced to engage the threaded rod 35 so that the tool 57 can used to rotate and advance the threaded rod 35. The port 51 may be placed as far anteriorly (in the front) as possible so that the inserted tool device 57 occupies the least amount of space within the chamber 21.

In operation, the fusion cage 10 of the present invention is in the collapsed position shown in FIG. 2 with the end 33 of the second spacer member 19 positioned immediately adjacent edge 43 of the first spacer member 15 when the cage is initially beginning to be inserted into the patient. The fusion cage 10 in this collapsed positioned is advanced into an incision made in the patient to position the fusion cage 10 between adjacent vertebrae in the spine, such as in a transforaminal lumbar spinal fusion procedure. As the fusion cage 10 is inserted it must move around the neural elements that are positioned adjacent the area where the fusion cage 10 will be located between the adjacent vertebrae. Essentially, the fusion cage 10 must be inserted and rotated around the neural elements to position the fusion cage in the desired location.

To reduce the intrusion of the fusion cage 10 into the body of the patient and to reduce the amount of displacement that may be necessary for the spinal cord it is desirable to articulate or bend the fusion cage so that it will more easily move around the spinal column. This becomes especially important when the fusion cage 10 is inserted through relatively small incisions utilizing an access tube. In such situations, there is little room for maneuverability, and a straight position of the cage during the initial insertion process is desirable. When the fusion cage 10 is inserted into the body so that the first spacer member 15 is extending past the dural sac, the tool 57 can be turned, much like a screwdriver, to advance the threaded rod 35 in the threaded passageway 31. The pivoting foot or ball in socket 47 on the end of the threaded rod 35 permits the edge 43 of the first spacer member 15 to be advanced away from the end 33 of the second spacer member 19 as the threaded rod 35 is advanced via operation of the tool 57. The advancement of the threaded rod 35 causes the first spacer member 15 to pivot away from second spacer member 19 around the pivot point or hinge 25 that connects the first spacer member 15 to the second spacer member 19. The threaded rod 35 is advanced until the first spacer member 15 is in the desired angular relationship with respect to the second spacer member 19 and the fusion cage 10 can be advanced into the patient in a direction that is less intrusive and not injurious to the body of the patient. The tool 57 can be used to adjust the angular position between the first spacer member 15 and the second spacer member 19 to facilitate the insertion of the fusion cage 10. As the first spacer member 15 is advanced between the adjacent vertebrae and around the spine, the threaded rod 35 can be advanced to increase the angle between the first spacer member 15 and the second spacer member 19. Increasing the angle allows the fusion cage 10 to progressively move to the angulated position so as to allow the fusion cage 10 to be positioned into the proper location between the adjacent vertebrae.

When the fusion cage 10 is fully inserted between the adjacent vertebrae, the threaded rod 35 will have been advanced so that the fusion cage 10 is in the angulated position shown in FIG. 1. When the fusion cage 10 has been angulated, the tool 57 can be disengaged from the threaded rod 35 and retracted until the threads in the tool 57 are engaged with the threads within port 51. The cage is then further advanced by using an impactor and properly located between the adjacent vertebrae. Tool 57 is then removed from the second spacer member 19. The end of the tool 57 that engages the threaded rod 35 will have a mechanism, as is well known in the art, to engage the threaded rod so that the tool can cause the threaded rod to be rotated in the threaded passageway 31. It will be appreciated that other advancement mechanisms for opening and collapsing the first and second spacer members 15 and 19, and other types of tools for selectively advancing the advancement mechanism are possible as well without departing from the spirit and scope of the present invention.

If desired, a shoulder (not shown) can be positioned in the threaded passageway 31 adjacent the opening 21 to act as a stop for the threaded rod 35. The shoulder will prevent the threaded rod 35 from being advanced into the opening 21 in the second spacer member 19.

The first and second spacer members 15, 19 of the cage 10 could be symmetric or asymmetric in size. The leading chamber 15 could be smaller (with 40:60 ratio with the trailing chamber 19). Such a configuration would decrease stresses on the leading chamber 15 as the tallest portion of the cage 10 would be located on the trailing chamber 19. This would, in turn, decrease the risk of shearing and stripping of the advancing mechanism 35.

If desired, the hinge 25 could be created with a scored metal rod. The hinge 25 is contained between the two chambers 15, 19, and the wings of the scored metallic rod are initially deployed to keep the cage 10 in a collapsed position. As the cage 10 is partially inserted, the wings of the scored metallic rod could be retracted allowing the rod to elongate between the two chambers 15, 19, which would angulate the cage.

While the present invention has been illustrated by a description of various embodiments and while these embodiments have been described in considerable detail, it is not the intention of the applicants to restrict or in any way limit the scope of the appended claims to such detail. Additional advantages and modifications will readily appear to those skilled in the art. The invention in its broader aspects is therefore not limited to the specific details, representative apparatus and method, and illustrative example shown and described. Accordingly, departures may be made from such details without departing from the spirit or scope of Applicant's general inventive concept.

Having described the invention, what is claimed is:

1. A cage to separate and support adjacent vertebrae in a spine, comprising:
a first spacer member for insertion between the adjacent vertebrae;
a second spacer member for insertion between the adjacent vertebrae;
an articulating mechanism located between said first and second spacers to connect the first spacer member to the second spacer member so that the first and second spacer members move relative to each other;
an advancing mechanism located between the first and second spacer members, the advancing mechanism being disposed to move the first and second spacer members relative to each other around the articulating mechanism wherein the angle of the first spacer member relative to the second spacer member facilitates the insertion of the cage around the spinal cord, the advancing mechanism being operable to position the first and second spacer members in a desired orientation relative to one another when the cage is fully positioned between the two adjacent vertebrae.

2. The cage of claim 1 wherein the articulating mechanism comprises a hinge.

3. The cage of claim 1 wherein the advancing mechanism comprises a rod that engages the cage.

4. A cage to separate and support adjacent vertebrae in a spine, comprising:
first and second spacer members mounted for articulation relative to each other and being configured for insertion between the adjacent vertebrae; and
an advancing mechanism located between the first and second spacer members, the advancing mechanism being operable to articulate the first and second spacer members relative to each other for insertion between the adjacent vertebrae.

5. A method for inserting a cage to separate and support adjacent vertebrae in a spine, the cage having first and second spacer members mounted for articulation relative to each other and an advancing mechanism operable to move the first and second spacer members relative to each other between collapsed and expanded positions, comprising:
inserting the cage between the adjacent vertebrae in the collapsed position wherein the first and second spacer members are positioned generally adjacent to each other; and
articulating the first and second spacer members relative to each other via operation of the advancing mechanism to configure the cage in the expanded position wherein the first and second spacer members are disposed at an angle relative to each other.
